(19) United States
(12) Patent Application Publication
(10) Pub. No.: US 2025/0239353 A1
(43) Pub. Date: Jul. 24, 2025

(54) AUTOMATIC RADIOTHERAPY PRESCRIPTION ANOMALY DETECTION

(71) Applicant: THE JOHNS HOPKINS UNIVERSITY, Baltimore, MD (US)

(72) Inventors: Todd R. MCNUTT, Severna Park, MD (US); Qiongge LI, Baltimore, MD (US); Michael BOWERS, Sparks, MD (US); Julie K. SHADE, Baltimore, MD (US); Theodore L. DEWEESE, Baltimore, MD (US); Andrew SHARABI, San Diego, CA (US)

(21) Appl. No.: 18/698,097

(22) PCT Filed: Oct. 6, 2022
(86) PCT No.: PCT/US2022/077664

§ 371 Date: Apr. 3, 2024

Related U.S. Application Data
(60) Provisional application No. 63/253,618, filed on Oct. 8, 2021.

(51) Int. Cl. G16H 20/40; A61N 5/10; G16H 10/60; G16H 50/70

(52) U.S. Cl. CPC G16H 20/40; A61N 5/1038; G16H 10/60; G16H 50/70

(57) ABSTRACT

Techniques for detecting that a radiotherapy prescription is anomalous are presented. The techniques include accessing historical patient data for historical patients, each including a historical radiotherapy prescription and a historical set of diagnostic features; determining a first measure or a second measure, where the first measure includes a distance between the radiotherapy prescription represented as a point in a first multidimensional space and a historical radiotherapy prescription represented as a point in the first multidimensional space, and where the second measure includes a distance between a set of diagnostic features of the patient represented as a point in a second multidimensional space and a historical set of diagnostic features, for a historical patient with a similar historical radiotherapy prescription, represented as a point in the second multidimensional space; detecting the first measure exceeding a first threshold or the second measure exceeding a second threshold; and issuing an alert.

### CROSS-REFERENCE TO RELATED APPLICATIONS

This application is the national stage entry of International Patent Application No. PCT/US2022/077664, filed on Oct. 6, 2022, and published as WO 2023/060168 A1 on Apr. 13, 2023, which claims priority to U.S. Provisional Patent Application No. 62/253,618, filed on Oct. 8, 2021, which are hereby incorporated by reference herein in their entireties.

### GOVERNMENT SUPPORT

This invention was made with government support under SBIR Phase II contract 2035750 awarded by the National Science Foundation. The government has certain rights in the invention.

### FIELD

This disclosure relates generally to prescription quality assurance.

### BACKGROUND

Appropriate dosage of radiation in radiotherapy is crucial in patient safety. Radiotherapy is a complex process that requires careful quality assurance to ensure safe treatment delivery. One such safety concern is with errant or uncommon prescriptions inadvertently being administered. Anomalies in prescription may occur for a variety of reasons. One possibility is a simple typographical error, such as the entering of 4×500 cGy by accident when the prescription 5×400 cGy is intended. While this type of human error is typically rare and is normally caught by multiple safety protocols that already exist, the impact of such an error could be clinically significant if not detected. Radiation dose calculation is nuanced and the biologic impact of different dose-per-fraction can be clinically significant, even when the cumulative dose is maintained. Especially important is that prescription errors in radiotherapy are particularly harmful as over-radiating the patient can lead to injury or death, whereas under-radiating the patient may fail to mitigate the cancer. Even though such errors are rare, the impact can range from sub-optimal treatment to catastrophe.

### SUMMARY

According to various embodiments, a method of detecting that a radiotherapy prescription for a patient is anomalous is presented. The method includes: accessing a plurality of historical patient data for a plurality of historical patients, each historical patient datum including a historical radiotherapy prescription and a historical set of diagnostic features; determining, by an electronic processor, at least one of a first measure or a second measure, where the first measure includes a distance between the radiotherapy prescription represented as a point in a first multidimensional space and a historical radiotherapy prescription represented as a point in the first multidimensional space, and where the second measure includes a distance between a set of diagnostic features of the patient represented as a point in a second multidimensional space and a historical set of diagnostic features, for a historical patient with a similar historical radiotherapy prescription, represented as a point in the second multidimensional space; detecting, by the electronic processor, at least one of the first measure exceeding a first threshold or the second measure exceeding a second threshold; and issuing an alert, by the electronic processor, in response to the detecting.

Various optional features of the above embodiments include the following. The method can include treating the patient according to a replacement prescription for the radiotherapy prescription. The method may include either: the similar prescription has identical fractions and doses per fraction to the new radiotherapy prescription, or the new radiotherapy prescription represented as a point in the first multidimensional space is closer to the similar historical radiotherapy prescription represented as a point in the first multidimensional space than to other historical radiotherapy prescriptions of the plurality of historical radiotherapy prescriptions represented as points in the first multidimensional space. The first

$$\frac{1}{m} {\sum }_{j ∈ m - closest} \sqrt{{( {f}_{i} - {f}_{j} )}^{2} + {( {d}_{i} - {d}_{j} )}^{2} ,}$$

where m is a number less measure may include than a number of the plurality of historical radiotherapy prescriptions, where ƒᵢ represents a number of fractions and dᵢ represents a dose per fraction of the new radiotherapy prescription, and where ƒᵢ represents a number of fractions and dⱼ represents a dose per fraction of a j-th historical radiotherapy prescription of the plurality of historical radiotherapy prescriptions. The first measure can include an average of distances between the radiotherapy prescription represented as a point in a first multidimensional space and multiple of the plurality of the historical radiotherapy prescriptions represented as points in the first multidimensional space. The second measure can include an average of distances between the set of diagnostic features of the patient represented as a point in a second multidimensional space and multiple historical sets of diagnostic features, each for a historical patient with a similar historical radiotherapy prescription, represented as points in the second multidimensional space. The second measure can include

$$\frac{1}{n} {\sum }_{j ∈ n - closest} g ( i , j ) ,$$

where n is a number less than a number of the plurality of historical radiotherapy prescriptions, where i represents the new radiotherapy prescription, and where j represents a j-th historical radiotherapy prescription of the plurality of historical radiotherapy prescriptions. The first threshold can include an average of distances between pairs of the plurality of historical radiotherapy prescriptions represented as points in the first multidimensional space. The first threshold can further include the average of distances weighted by a model parameter. The second threshold can include an average of distances between pairs of historical sets of diagnostic features of the plurality of historical patients represented as points in the second multidimensional space. The second threshold can further include the average of distances weighted by a model parameter. The method can include: generating simulated anomalous patient data, the simulated anomalous patient data including at least one of: a simulated radiotherapy prescription mismatched with a set of diagnostic features, or a simulated radiotherapy prescription that occurs in less than a predetermined proportion of the plurality of historical patient data; and optimizing model parameters using the simulated anomalous patient data. The method can include: determining that an element of the radiotherapy prescription occurs in less than some predetermined proportion of the historical radiotherapy prescriptions for a same technique as the radiotherapy prescription; and issuing an alert, by the electronic processor, in response to the determining that an element of the radiotherapy prescription occurs in less than some predetermined proportion of the historical radiotherapy prescriptions for a same technique as the radiotherapy prescription. The set of diagnostic features can include: treatment technique, treatment energy, treatment intent, diagnostic code, morphology code, and patient age. The issuing the alert can include one of: issuing an alert indicating that the radiotherapy prescription is unlike historical radiotherapy prescriptions, or issuing an alert indicating that the radiotherapy prescription is mismatched to the diagnostic features of the patient. The issuing the alert can include displaying the alert on a computer monitor.

According to various embodiments, a system for detecting that a radiotherapy prescription for a patient is anomalous is presented. The system includes an electronic processor and non-transitory electronic persistent storage storing instructions that, when executed by the electronic processor, perform actions including: accessing a plurality of historical patient data for a plurality of historical patients in non-transitory electronic persistent storage, each historical patient datum including a historical radiotherapy prescription and a historical set of diagnostic features; determining, by the electronic processor, at least one of a first measure or a second measure, where the first measure includes a distance between the radiotherapy prescription represented as a point in a first multidimensional space and a historical radiotherapy prescription represented as a point in the first multidimensional space, and where the second measure includes a distance between a set of diagnostic features of the patient represented as a point in a second multidimensional space and a historical set of diagnostic features, for a historical patient with a similar historical radiotherapy prescription, represented as a point in the second multidimensional space; detecting, by the electronic processor, at least one of the first measure exceeding a first threshold or the second measure exceeding a second threshold; and issuing an alert, by the electronic processor, in response to the detecting.

Various optional features of the above embodiments include the following. The first measure can include an average of distances between the radiotherapy prescription represented as a point in a first multidimensional space and multiple of the plurality of the historical radiotherapy prescriptions represented as points in the first multidimensional space. The first measure may include

$$\frac{1}{m} {\sum }_{j ∈ m - closest} \sqrt{{( {f}_{i} - {f}_{j} )}^{2} + {( {d}_{i} - {d}_{j} )}^{2} ,}$$

where m is a number less than a number of the plurality of historical radiotherapy prescriptions, where ƒᵢ represents a number of fractions and dᵢ represents a dose per fraction of the new radiotherapy prescription, and where ƒⱼ represents a number of fractions and dⱼ represents a dose per fraction of a j-th historical radiotherapy prescription of the plurality of historical radiotherapy prescriptions. The second measure can include an average of distances between the set of diagnostic features of the patient represented as a point in a second multidimensional space and multiple historical sets of diagnostic features, each for a historical patient with a similar historical radiotherapy prescription, represented as points in the second multidimensional space. The second measure can include

$$\frac{1}{n} {\sum }_{j ∈ n - closest} g ( i , j ) ,$$

where n is a number less than a number of the plurality of historical radiotherapy prescriptions, where i represents the new radiotherapy prescription, and where j represents a j-th historical radiotherapy prescription of the plurality of historical radiotherapy prescriptions. The first threshold can include an average of distances between pairs of the plurality of historical radiotherapy prescriptions represented as points in the first multidimensional space. The first threshold can further include the average of distances weighted by a model parameter. The second threshold can include an average of distances between pairs of historical sets of diagnostic features of the plurality of historical patients represented as points in the second multidimensional space. The second threshold can further include the average of distances weighted by a model parameter. The actions can further include: generating simulated anomalous patient data, the simulated anomalous patient data including at least one of: a simulated radiotherapy prescription mismatched with a set of diagnostic features, or a simulated radiotherapy prescription that occurs in less than a predetermined proportion of the plurality of historical patient data; and optimizing model parameters using the simulated anomalous patient data. The actions can further include: determining that an element of the radiotherapy prescription occurs in less than some predetermined proportion of the historical radiotherapy prescriptions for a same technique as the radiotherapy prescription; and issuing an alert, by the electronic processor, in response to the determining that an element of the radiotherapy prescription occurs in less than some predetermined proportion of the historical radiotherapy prescriptions for a same technique as the radiotherapy prescription. The set of diagnostic features can include: treatment technique, treatment energy, treatment intent, diagnostic code, morphology code, and patient age. The issuing the alert can include one of: issuing an alert indicating that the radiotherapy prescription is unlike historical radiotherapy prescriptions, or issuing an alert indicating that the radiotherapy prescription is mismatched to the diagnostic features of the patient. The system can include a computer monitor that issues the alert by displaying the alert on the computer monitor.

According to various embodiments, a non-transitory computer-readable medium including instructions that, when executed by an electronic processor, configure the electronic processor to detect that a radiotherapy prescription for a patient is anomalous by performing actions is disclosed. The action include: accessing a plurality of historical patient data for a plurality of historical patients, each historical patient datum including a historical radiotherapy prescription and a historical set of diagnostic features; determining, by an electronic processor, at least one of a first measure or a second measure, where the first measure includes a distance between the radiotherapy prescription represented as a point in a first multidimensional space and a historical radiotherapy prescription represented as a point in the first multidimensional space, and where the second measure includes a distance between a set of diagnostic features of the patient represented as a point in a second multidimensional space and a historical set of diagnostic features, for a historical patient with a similar historical radiotherapy prescription, represented as a point in the second multidimensional space; detecting, by the electronic processor, at least one of the first measure exceeding a first threshold or the second measure exceeding a second threshold; and issuing an alert, by the electronic processor, in response to the detecting.

### BRIEF DESCRIPTION OF THE DRAWINGS

Various features of the embodiments can be more fully appreciated, as the same become better understood with reference to the following detailed description of the embodiments when considered in connection with the accompanying figures, in which:

FIG. 1 is a graph illustrating the number of occurrences by radiation therapy techniques used in historical thoracic patients;

FIG. 2 is a graph illustrating energy used in 3D radiation therapy;

FIG. 3 depicts a schematic diagram of a reduction to practice;

FIG. 4 depicts a density plot of BED, a density plot of fractions, and a density plot of dose-per-fraction for different techniques;

FIG. 5 depicts two different anomalous cases the reduction to practice model is designed to catch in an illustrative 3D feature space;

FIG. 6 depicts a decision tree for the logic of the model according to the reduction to practice;

FIG. 7 shows normalized histograms of Rx and feature distances in the historical patients' database;

FIG. 8 depicts the results of a mock peer review of the reduction to practice; and

FIG. 9 shows Venn diagrams illustrating the overlap of agreement between individual MDs, the MD's consensus, the model of the reduction to practice, and the ground truth.

### DESCRIPTION OF THE EMBODIMENTS

Embodiments as described herein are described in sufficient detail to enable those skilled in the art to practice the invention, and it is to be understood that other embodiments may be utilized and that changes may be made without departing from the scope of the invention. The descriptions are, therefore, merely exemplary.

### I. Introduction

Embodiments may be used to detect whether a radiotherapy prescription is anomalous. For example, embodiments may detect an anomaly in the form of parameters of a radiotherapy prescription (e.g., number of radiotherapy dose fractions and dose per fraction) for patient being mismatched with diagnostic features for the patient (e.g., any combination of treatment technique, treatment energy, treatment intent, diagnostic code, morphology code, patient age, and treatment intent). As another example, embodiments may detect an anomaly in the form of a radiotherapy prescription for a patient that is unlike any historical radiotherapy prescription for known historical patients. Thus, some embodiments can detect anomalies of at least the two above-described types.

Some embodiments improve patient safety through the use of a prescription anomaly detection tool that implements an automated, historical data-driven checkpoint to assist in peer review. The tool defines distance metrics between a new patient's features and prescriptions and those in a historical database. According to some embodiments, the treatment technique and energy may be considered as fixed features rather than part of the prescription. According to some embodiments, the elements of the prescription are considered the dose per fraction and number of fractions prescribed to the target volume. Besides prescription features, there are other features such as diagnosis code, age at treatment, disease stage, treatment intent. Using a logical rule-based approach, the tool flags the new patient's prescription as anomalous if the distances fall outside certain optimized thresholds within a subgroup of similar patients.

In general, anomaly detection is a hard problem for a data-driven approach because of the lack of anomaly data with which to train a traditional machine learning algorithm. Some embodiments have the advantage of using very little data due to an imposed separation between prescription features (e.g., number of fractions and dose per fraction) and diagnostic features. Thus, some embodiments do not need a lot of data compared to traditional machine learning models (such as random forests, neural networks, etc.). For example, the mock peer review described herein in Section VI proves some embodiments' effectiveness using just thirty anomaly samples to optimize the model parameters.

Another reason why anomaly detection is challenging in general is because there is no clear definition of what can be called an anomaly, especially in medicine (unlike, e.g., credit card fraud). However, some embodiments utilize a clear definition of what can be considered an anomaly, e.g., the two types of anomalies presented above and further described herein.

Because of the reasons described above, as well as other reasons, practical anomaly detection in the medical field (not just radiotherapy) is rare.

Some embodiments advantageously use a rule based algorithm rather than a machine learning algorithm such as neural network. This approach is superior to machine learning for anomaly detection, because it can explain the reason why a particular data point is flagged as anomalous. By contrast, some machine learning algorithms are “black box” and do not explain why they predict a particular result. Pure machine learning algorithms also tend to have difficulty with anomaly detection since the power of machine learning is from big data where the anomalous class is rare and few data points exist to learn from. Class balancing methods can be used, but do not always address the lack of information about the rare class. This can present a major difficulty for supervised learning.

Another problem with supervised machine learning approaches to anomaly detection is that the prescription features belong to a separate class distinct from the diagnostic features. The diagnosis could be for a very rare condition, however, that is not an anomaly according to various embodiments. Rather, an anomaly may be characterized as a situation where the prescription does not match the diagnosis, e.g., either an error has occurred in the prescription or the recording of other features so that there is a mismatch in the sets of prescriptions and diagnostic features. For a hypothetical supervised learning model to make the separation between prescription and diagnostic features would require a lot of data, data which is difficult to obtain or generate. A rule based model can impose the separation between the prescription and diagnostic features instead having to learn it.

Thus, compared to traditional machine learning algorithms, some embodiments have the advantage of requiring little data to optimize the model. In addition, because some embodiments utilize a non-traditional data-driven approach, such embodiments include explanation power (e.g., providing a reason for a new radiotherapy prescription being considered an anomaly). Traditional machine learning techniques typically lack this capacity.

Some embodiments provide a multi-layer anomaly detection tool that is fully automatic so that no human time is needed to run the algorithm. Thus, some embodiments may serve as an extra safeguard augmenting the peer review process.

These and other features and advantages are presented in detail herein.

### II. Data Description

A reduction to practice is described in detail throughout this disclosure. A description of the data used in the reduction to practice follows.

The reduction to practice utilized fifteen years of cancer patients' radiotherapy treatment data (Jan. 1, 2006-Jul. 13, 2021) from MOSAIQ (a radiation oncology-specific electronic medical record). The data was included 63,768 individual treatment prescriptions, which includes all the patients treated in the radiation oncology department of Johns Hopkins University Hospital (with all the campuses) over the time span. Features related to patients' treatment information were extracted, including patient's age at treatment, diagnosis code, morphology code, treatment intent, techniques, energy, anatomic site, tumor stages (T, N, M stages), tumor markers, and biomarkers. The total number of features in the raw dataset was 33.

Prescription (Rx) data includes the number of fractions, dose per fraction, total dose, and accumulated total dose. The patients were grouped by disease site including thoracic, central nervous system, head and neck, prostate, and breast based on diagnostic codes.

### III. Initial Analysis and Feature Engineering

The inventors conducted exploratory data analysis (EDA) to understand important patterns in the data.

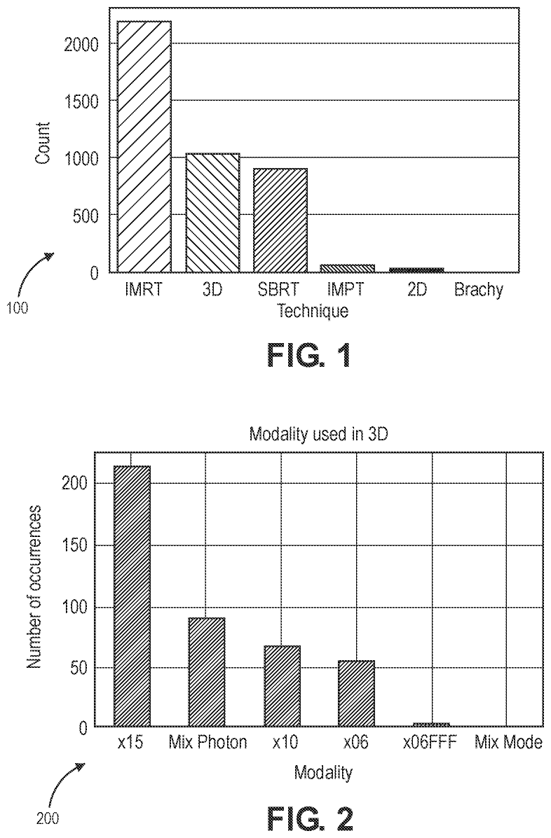

FIG. 1 is a graph 100 illustrating the number of occurrences (in percentages) by radiation therapy techniques used in historical thoracic patients. Tomotherapy was included under Intensity-modulated radiation therapy (IMRT) because it is a sub-class of IMRT. As shown in FIG. 1, IMRT, which occurred 2195 times in the database, 3D, which occurred 1040 times, and SBRT, which occurred 903 times, were the top the most popular used techniques in treating thoracic patients. There were not enough samples to build models for the following treatment techniques: Intensity modulated proton therapy (IMPT), two dimensional basic radiotherapy (2D) and Brachytherapy (Brachy). Therefore, these techniques were not considered in the subsequent analysis. However, additional embodiments may include such techniques if sufficient data is present. The techniques that were kept for later analysis according to the reduction to practice were three-dimensional conformal radiation therapy (3D), IMRT, and stereotactic body radiotherapy (SBRT).

FIG. 2 is a graph 200 illustrating energy used in 3D radiation therapy. As shown in FIG. 2, “Mix Mode” and “x06FFF” were rarely used for 3D. Similar analysis shows that “x15” and “x10FFF” were rarely used for IMRT, and that for SBRT only “x06” and “x06FFF” were commonly used. Highly rare energies for each technique were removed from the historical data sets.

A number of feature engineering steps were used to transform the columns into relevant form or to remove columns (features) that were not relevant. Natural language processing (NLP) was used to remove columns (converge many similar labels to a single values). In other cases, irrelevant features were removed. For example, Gleason scores were helpful for prostate cancer, but irrelevant to the thoracic group cancer.

Several composite prescription variables were also built. The two numerical features, number of fractions and dose per fraction, were combined into a categorical string. For example, the Rx ‘10×300’ creates a single variable that describes both fractions and dose per fraction. The Biological Effective Dose (BED) was also calculated:

$$B E D = d \times f \times ( 1 + d / 10 ) \qquad ( 1 )$$

In Equation (1), fis the number of fractions and d is the dose per fraction. BED serves as an alternative composite variable that characterizes the cell damage effect of the prescription. Example values of these variables are shown in Table I.

TABLE 1

| | | | | | | | | | | Targets | | | | | |
|---|---|---|---|---|---|---|---|---|---|---|---|---|---|---|---|
| Features | | | | | | | | | | | | | Cum | | |
| Tx | Tx | | | | | | | | | | | Ttl | Ttl | | |
| Age | Site | Technique | Energy | Intent | ICD10 | ICDO | T | N | M | Fx | Dose/fx | Dose | Dose | Rx | BED |
| 63 | lul | sbrt | x06 | Palliative | C34.10 | 81406 | 2 | 1 | 1 | 4 | 1200 | 4800 | 4800 | 4 × 1200 | 580800 |
| | nodule | | | | | | | | | | | | | | |

The feature set for technique was reduced to be 3D, IMRT and SBRT and then mapped the energy to be x06, x10, x15 and mixed photon. The raw data was also mapped into the following treatment intent: curative or palliative.

A unique list of diagnosis codes and their description was created and validated by the physicians. The completeness and appropriateness of the diagnosis codes for the model was confirmed; the values are shown in the Table 2 for the thoracic. The reduction to practice only included cancer patients whose primary tumor site was lung, heart, or esophagus. Liver and stomach cancer was excluded from this model.

TABLE 2

| Diagnosis | |
|---|---|
| code | |
| (ICD10) | Description of the code |
| C15.3 | Malignant neoplasm of upper third of esophagus |
| C15.4 | Malignant neoplasm of middle third of esophagus |
| C15.5 | Malignant neoplasm of lower third of esophagus |
| C15.9 | Malignant neoplasm of esophagus, unspecified |
| C33 | Malignant neoplasm of trachea |
| C34.00 | Malignant neoplasm of unspecified main bronchus |
| C34.01 | Malignant neoplasm of right main bronchus |
| C34.02 | Malignant neoplasm of left main bronchus |
| C34.10 | Malignant neoplasm of upper lobe, unsp bronchus or lung |
| C34.12 | Malignant neoplasm of upper lobe, left bronchus or lung |
| C34.2 | Malignant neoplasm of middle lobe, bronchus or lung |
| C34.30 | Malignant neoplasm of lower lobe, unsp bronchus or lung |
| C34.31 | Malignant neoplasm of lower lobe, right bronchus or lung |
| C34.32 | Malignant neoplasm of lower lobe, left bronchus or lung |
| C34.80 | Malignant neoplasm of ovrlp sites of unsp bronchus and |
| | lung |
| C34.81 | Malignant neoplasm of ovrlp sites of right bronchus and |
| | lung |
| C34.82 | Malignant neoplasm of ovrlp sites of left bronchus and |
| | lung |
| C34.90 | Malignant neoplasm of unsp part of unsp bronchus or lung |
| C34.91 | Malignant neoplasm of unsp part of right bronchus or lung |
| C34.92 | Malignant neoplasm of unsp part of left bronchus or lung |
| C37 | Malignant neoplasm of thymus |
| C38.1 | Malignant neoplasm of anterior mediastinum |
| C38.2 | Malignant neoplasm of posterior mediastinum |
| C38.3 | Malignant neoplasm of mediastinum, part unspecified |
| C38.4 | Malignant neoplasm of pleura |
| C38.8 | Malignant neoplasm of ovrlp sites of heart, mediastinum |
| | and pleura |
| C45.0 | Mesothelioma of pleura |
| C77.1 | Secondary and unsp malignant neoplasm of intrathorac |
| | nodes |
| C78.00 | Secondary malignant neoplasm of unspecified lung |
| C78.01 | Secondary malignant neoplasm of right lung |
| C78.02 | Secondary malignant neoplasm of left lung |
| C78.1 | Secondary malignant neoplasm of mediastinum |
| C78.2 | Secondary malignant neoplasm of pleura |
| D15.0 | Benign neoplasm of thymus |
| E85.8 | Other amyloidosis |
| R91.1 | Solitary pulmonary nodule |

Re-plans and cone-down plans with their initials were searched for by finding the mismatch of the total dose and the total accumulated dose. Because they are only 2.6% of the total data points, these patients' re-plan treatment along with their initial treatment were eliminated. The inventors also searched for keywords pertaining to conedown and eliminated those records.

A number of additional checks were performed filter out atypical/strange data (e.g., samples with a total dose that does not match with fractions times the dose per fraction). Eventually, 2356 rows of records for thoracic were acquired. See Table I, which shows a sample post processed feature-set.

### IV. Reduction to Practice Model

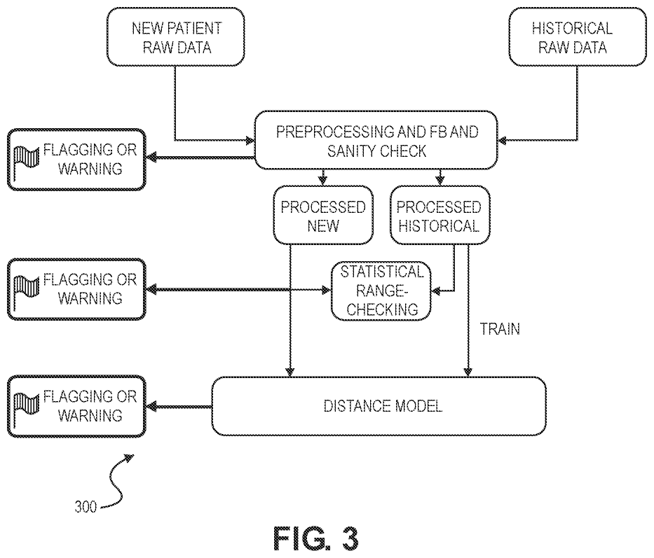

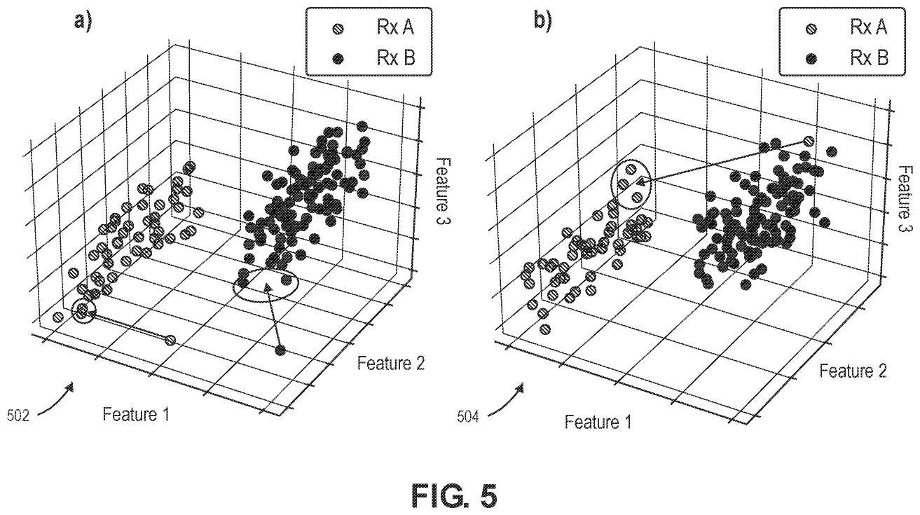

FIG. 3 depicts a schematic diagram 300 of the reduction to practice. The model compares a new patient's prescriptions and other features to those in a historical database and flags any suspicious patterns because they have not been previously seen or are rare. Both the historical data and the new patient's data are processed as described herein in Section III. The range checking and distance model components of the pipeline and under what circumstances the new patient's prescription will be flagged as a potential anomaly are described herein in reference to FIGS. 5-7.

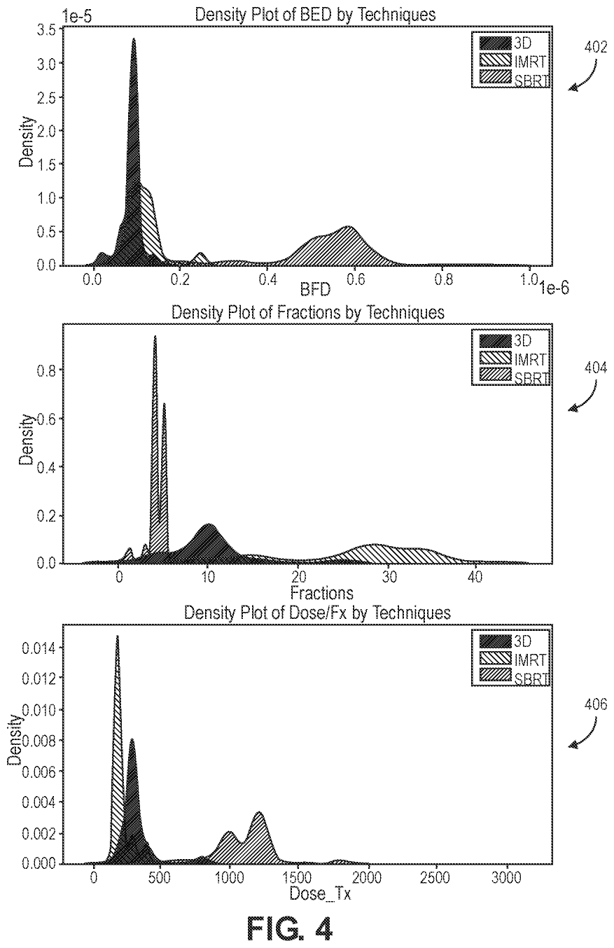

FIG. 4 depicts a density plot of BED 402, a density plot of fractions 404, and a density plot of dose-per-fraction 406 for different techniques. Different shades stand for different techniques. The fitted continuous density curves provide insight as to how rare a prescription feature is. These data are used check whether the numerical values of a new patient's prescription are within historical normal ranges. The distribution curves of FIG. 4 are used to determine boundaries of normal prescriptions. Then, any new patient's prescription may be flagged if it falls outside these boundaries.

According to various embodiments, and per the reduction to practice, a distance model defines a logical system that is used to flag the new patient if its distance from other patients in the historical database, or specific groups of patients in the historical database, is too large. The model detects the following two types of prescription anomalies: the Rx itself is atypical from the historical records (Type 1 anomaly) and there is a mismatch between Rx and patients' other diagnostic features (Type 2 anomaly). In order to compare the new patient's prescription and other features with patients in the historical database, pairwise and group level dissimilarity metrics are defined. Thus, two such distance metrics are presented: a prescription distance to indicate the distance in the prescription parameters, and a feature distance to indicate distance within the remaining features included in the model.

The pairwise Rx distance, ρ_{Rx}(i,j) between the new patient, i, to any historical patient, j, in the database, may be represented as the Euclidean distance of the scaled prescription features, by way of non-limiting example, as follows:

$${ρ}_{R x} ( i , j ) = \sqrt{{( {\tilde{f}}^{i} - {\tilde{f}}^{j} )}^{2} + {( {\tilde{d}}^{i} - {\tilde{d}}^{j} )}^{2}} \qquad ( 2 )$$

In Equation (2), \tilde{ƒ} and \tilde{d} represent the min-max scaled fraction ƒ and dose per fraction d, respectively.

The pairwise feature distance, g_{F}(i,j), between the new patient, i, and any historical patient, j, in the database may be represented as the Gower distance calculated over features that are not prescription-related. In general, Gower distance provides a way of computing dissimilarity when mixed numerical and categorical features are present. Numerical features contribute based on the absolute value of the difference divided by the range. For categorical features, the dissimilarity is one if they are different and zero if they are the same. Each feature in the Gower distance is given equal weight so that the Gower metric has a range on the interval [0,1].

In addition to pairwise dissimilarity metrics, some embodiments may utilize a “closest-m group distance” of the new patient i, R(i,m), which may be represented as the average of the m shortest Rx distances between patient i and patient's j in the historical data, by way of non-limiting example, as follows:

$$R ( i , m ) = \frac{1}{m} {\sum }_{j ∈ m - c losest} {ρ}_{R x} ( i , j ) \qquad ( 3 )$$

Similarly, some embodiments may utilize a “closest-n group distance”, F(i), for all non-prescription related features that applies the same formula but sums over n pairwise Gower distances between the new patient, i and patients, k, in the historical database, which may be represented as follows, by way of non-limiting example:

$$F ( i , n ) = \frac{1}{n} {\sum }_{k ∈ n - closest} {g}_{F} ( i , j ) \qquad ( 4 )$$

In Equation (4), n terms are determined by sorting by ρ_{Rx}(i,j) then by g_{F}(i,j). Further, Equation (4) restricts the sum to patients k who have either the same prescription as patient i or who have minimal Rx distance to patient i. For example, if n=10 and there are 12 patients with the same Rx as patient i in the historical then select the lowest 10 Gower distances from this group of 12. If n=20, then first include all 12 terms ρ_{Rx}(i,j)=0 in the sum to compute F and then sort over the next closest Rx distance to find remaining terms in a similar fashion. This metric may be utilized because features are expected to be more similar when compared to others with the same (or similar) prescription.

In order to define thresholds that will define the cutoff for flagging, it is useful to calculate some characteristic values of pairwise distances in the historical dataset. In this way, two patient's features can be precisely defined as similar or dissimilar. They can be characterized as dissimilar if their feature distance is much larger than the average historical pairwise distances for two patient's with the same Rx. The mean pairwise Rx distance and the mean pairwise feature distance are computed over all pairs of patients in the historical database to get a typical distance, θ and τ, which may be represented as follows, by way of non-limiting example:

$$θ = \frac{1}{S ( S - 1 )} {\sum }_{j , k} {ρ}_{R x} ( j , k ) \qquad ( 5 )$$

$$τ = \frac{1}{S ( S - 1 )} {\sum }_{j , k} {g}_{F} ( j , k ) \qquad ( 6 )$$

In Equations (5) and (6), S is the number of patients in the historical data base and, again, ρ_{Rx}(j,k), g_{F}(j,k) are distances between a pair of historical patients j and k.

According to various embodiments, the thresholds may be patterned as percentages of these characteristic values as follows, by way of non-limiting example:

$${t}_{R x} = a θ \qquad ( 7 )$$

In Equation (7), a is a model parameter that may be determined by optimization. If R>t_{Rx} then the corresponding prescription may be flagged as an anomaly (Type 1). Similarly, the feature threshold may be represented as a ratio of some characteristic values as follows, by way of non-limiting example:

$${t}_{F} = b τ \qquad ( 8 )$$

In Equation (8), b is a model parameter, which may be determined by optimization. If F>t_{F} then the corresponding prescription may be flagged as an anomaly (Type 2).

FIG. 5 depicts two different anomalous cases 502, 504 the model is designed to catch in an illustrative 3D feature space. The first case 502 illustrates two feature anomalies that are far from the average. The second case 504 illustrates Rx A is mismatched within the feature sector of Rx B. In both cases, anomalies can be detected if they are far away from the n-group centroids belonging to their Rx. Note that in FIG. 5, the n-group centroids are determined by the data points on the surface of the Rx cluster closest to each anomaly data point. In case 502, the anomalies are isolated in the feature space, whereas in case 504, a single anomaly is mismatched into an incorrect Rx sector of the feature space.

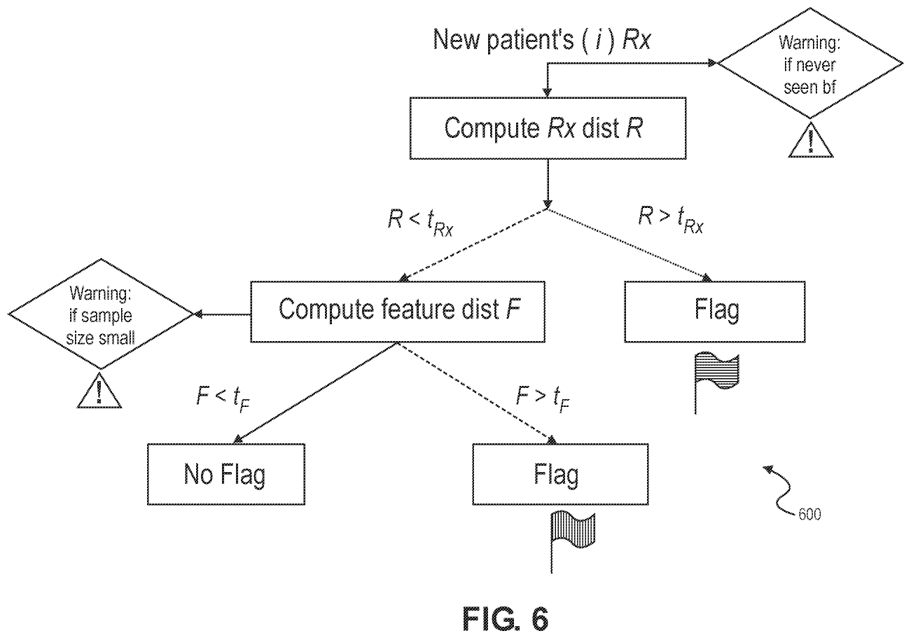

FIG. 6 depicts a decision tree 600 for the logic of the model according to the reduction to practice. The reduction to practice used dissimilarity metrics R and F to flag an incoming new patient. The closest-m Rx group distance, R(m), is computed, and the Rx is flagged if it is larger than some threshold t_{Rx}. If R is too large (e.g., R is greater than t Rx), then the new patient's prescription is too dissimilar when considering other prescriptions in the historical database and it is flagged. Otherwise, if R<t_{Rx}, then the closest-n group feature distance F is computed, considering only patients with the same Rx as patient i. A warning is given if there does not exist n patients in the historical database with the same prescription as the new patient, i. If F is more than the threshold t_{F} then the Rx is flagged for the new patient as a mismatch between the prescription and their other features, at for the data in the historical database.

A description of training the model of the reduction to practice follows. The model includes four parameters: m, n, a, and b. In order to scale with the size of the historical dataset, the parameters m, n, are re-expressed as percentages of the historical training set size. Thus m=μS, where S is the number of samples in the historical database per technique after subtracting a holdout set, and μ is the parameter we use for hyperoptimization. Similarly, let n=νS and optimize of over the percentage ν. Thus, the final set of parameters for optimization are μ, ν, a and b.

The reduction to practice made use of a parameter space search (grid-search) optimization to determine these parameters. The objective function for optimization was taken as the f1 score over a training set that includes 10-30 simulated anomalies and a similar number of non-anomalous patients. Thus, the training set included simulated anomalies as well as holdout data from the historical database so that as to include both positive (anomaly) and negative (not anomaly) classes in the test set.

Optimization through parameter space search was implemented with the python hyperopt module. Hyperopt uses the tree Parzen Estimator (TPE) to efficiently search the parameter space. Search intervals were defined based on the characteristic values 0 and t for parameters a and b. Search intervals for the percentages u and ν were constrained to be between 0 and 0.1, which confines the m,n-group dissimilarity metrics to 10% of the historical database or lower for calculations of F and R. The number of evaluations was set to 100 per each space search of the detection algorithm.

In order to reduce variance in the normal (not-anomaly) class, the results were averaged over random samplings of the non-anomalous holdout historical records. During this averaging, the anomaly class data points remained constant due to the fact that there were a limited number of simulated anomalies available for training.

Simulated anomalies were based on distributions. Creation of the anomalies is a time consuming task that includes careful examination of the historical database and identification of non-previously-occurring patterns between prescription and other features. The construction is illustrated presently with some examples below. The main idea is to change the prescription of an existing record, or to change the other features of an existing record, in a way that creates a data point that not typical of historical prescription-feature patterns. In this way a mismatch between the prescription and the other features is created. This mismatch is verified by observing conditional distributions of features based on the given Rx for each case. Thus, the anomalies constructed are rare based on the historical conditional distributions.

The simulated anomalies are constructed so as to be similar to those that could occur in the real setting. By carefully designing the anomalies, the correct parameters to generalize the model's application to the real world can be obtained. The model parameters can be tuned so as to catch each of the simulated anomalies and flag them.

Simulated anomalies were generated by switching the leading digit in the fractions with the leading digit in the dose per fraction or by varying several feature values randomly in such a way that the resulting features do not match the prescription. Table III shows four examples, marked A-D, where the original record is placed above its anomalous mutated form. In example A, the fractions (Fx) and dose per fraction (Dose/Fx) were switched from 5×400 to 4×500. 5×400 is a common prescription in 3D thoracic treatment having occurred 50 times in the historical database, but not 4×500 which occurred only once.

TABLE 3

| | | Tx | | Dose/ | | | Tx | | | |
|---|---|---|---|---|---|---|---|---|---|---|
| Example | | Site | Fx | Fx | Tech. | Modal. | Intent | ICD 10 | ICDO | Age |
| A | orig | rt | 5 | 400 | 3D | mixed | | C34.90 | 80463 | 76 |
| | | clavicle | | | | photon | | | | |
| | mutate | rt | 4 | 500 | 3D | mixed | | C34.90 | 80463 | 76 |
| | | clavicle | | | | photon | | | | |
| B | orig | lul | 5 | 1000 | SBRT | x06fff | curative | R91.1 | | 91 |
| | | central | | | | | | | | |
| | | module | | | | | | | | |
| | mutate | lul | 5 | 1000 | SBRT | x06fff | palliative | R91.1 | | 10 |
| | | central | | | | | | | | |
| | | module | | | | | | | | |
| C | orig | ptv III lt | 4 | 1200 | SBRT | x06 | pallitive | C34.30 | 87203 | 49 |
| | | lung les | | | | | | | | |
| | mutate | ptv III lt | 4 | 1200 | SBRT | x10fff | pallitive | C15.9 | 87203 | 49 |
| | | lung les | | | | | | | | |
| D | orig | left sup | 5 | 400 | SBRT | x06 | | C34.10 | 91803 | 13 |
| | | pleural | | | | | | | | |
| | | ptv | | | | | | | | |
| | mutate | left sup | 5 | 400 | 3D | x06 | | C34.10 | 91803 | 13 |
| | | pleural | | | | | | | | |
| | | ptv | | | | | | | | |

In B and C, the simulated anomalies were created by modifying other features and leaving the original prescription intact. For example, in case B, the treatment intent was changed from curative to palliative and the age from 91 to 10. The prescription 5×1000 occurred 185 times in SBRT thoracic treatment but never occurred with palliative intent. Also, this Rx was never used in a pediatric patient (age under 21). Thus the features were varied in a way that created a mismatch between prescription and diagnostic features. In C, the diagnostic code was mutated from C34.30 to C15.9. Comparing with the historical records, this Rx never treated the esophagus (which has a diagnostic code in the C15 series), and only was used to treat the lungs (C34 series). Also, the energy was mutated from x06 to x10, which never occurred for this Rx.

In the last example D, an anomaly was simulated by switching the technique label from SBRT to 3D, so that effectively all the features are mismatched. 5×400 is a common Rx in 3D (occurring 50 times), but a rare Rx for SBRT. The feature sets are quite distinct, because in 3D, the energy that comes with this Rx is usually 15×, but 15× never occurred in historical SBRT cases with this Rx.

It should be noted that this approach to simulating anomalies is purely data driven and based on deviations from past historical patterns. The anomaly creation process was done by authors with no clinical information (authors who are MDs were excluded from this process).

### V. Results From the Reduction to Practice

This section provides illustrative results from the reduction to practice for the thoracic group.

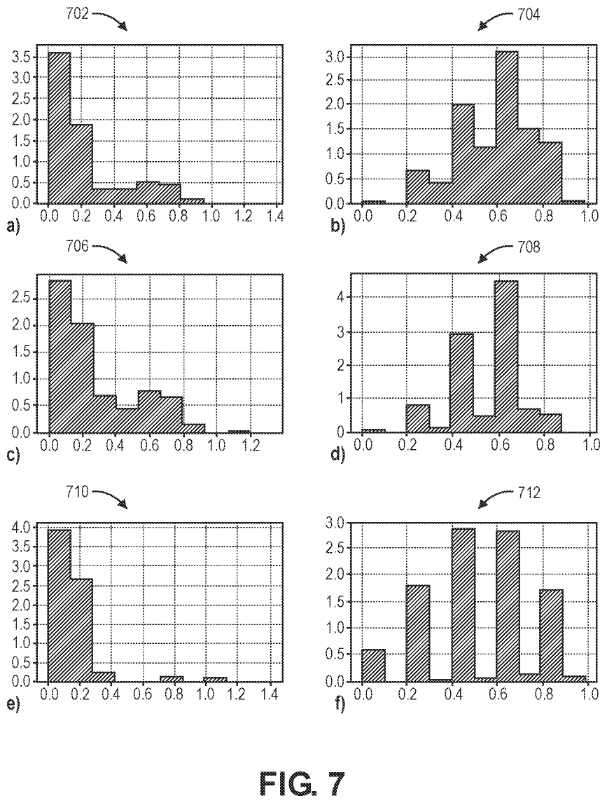

FIG. 7 shows normalized histograms 702, 704, 706, 708, 710, 712 of Rx and feature distances in the historical patients' database. Histograms 702 and 704 are for 3D, histograms 706 and 708 are for IMRT, and histograms 710 and 712 are for SBRT. Histograms 702, 706, 710 are for the distributions of pairwise prescription distance ρ_{Rx}(j,k), and histograms 704, 708, and 712 are for the distributions of Gower feature distance g_{F}(j,k) where j, k are a historical patients' pair. Spikes relate to the three categorical features through the dice similarity. If one categorical feature is different, the net dissimilarity is 0.2; if two categoricals are different, it is 0.4 etc. In SBRT, the pairwise Gower distances are dominated by categorical features.

In the histograms 702, 704, 706, 708, 710, 712 of FIG. 7, the Rx distances of zero or 0.2 are particularly common, which reflects the fact that many patients in the dataset have the same prescription. The feature distances are more varied and only a smaller subset of the patients share the same features.

As discussed above, there are several ways in which anomalies were synthesized. Table 4 presents the in-sample training results for the Rx switched (see Example A in Table III) type of simulated anomalies. The S column refers to the number of records in the historical database, a,b are the parameters multiplying θ and τ, respectively, and μ=m/S and =n/S are the parameters m and n expressed as percentages of S. Sa refers to the number of anomalies in the training set whereas sₙ refers to the number of normal holdout historical samples in the training set. Note that the holdout set sₙ is not used to compute θ or τ.

TABLE 4

| | Tech. | a | b | v | μ | τ | θ | f1 | sₙ | sₐ | S |
|---|---|---|---|---|---|---|---|---|---|---|---|
| Rx | 3D | 0.449 | 1.632 | 0.012 | 0.018 | 0.581 | 0.206 | 0.98 ± 0.03 | 20 | 10 | 509 |
| switched | IMRT | 0.265 | 0.979 | 0.025 | 0.014 | 0.543 | 0.261 | 0.89 ± 0.01 | 20 | 10 | 1153 |
| SAs | SBRT | 1.631 | 1.838 | 0.047 | 0.014 | 0.501 | 0.142 | 0.98 ± 0.03 | 20 | 10 | 704 |
| Feature | 3D | 0.056 | 0.797 | 0.021 | 0.019 | 0.581 | 0.206 | 0.84 ± 0.02 | 20 | 20 | 509 |
| switched | IMRT | 0.286 | 0.802 | 0.023 | 0.038 | 0.543 | 0.261 | 0.84 ± 0.01 | 20 | 20 | 1153 |
| SAs | SBRT | 0.307 | 0.584 | 0.017 | 0.029 | 0.501 | 0.142 | 0.90 ± 0.03 | 20 | 20 | 704 |
| In-sample | 3D | 0.010 | 0.717 | 0.010 | 0.037 | 0.581 | 0.206 | 0.84 ± 0.01 | 30 | 30 | 499 |
| (both types | IMRT | 1.401 | 0.805 | 0.025 | 0.014 | 0.543 | 0.261 | 0.86 ± 0.01 | 30 | 30 | 1143 |
| of SAs) | SBRT | 1.926 | 0.465 | 0.01 | 0.075 | 0.501 | 0.142 | 0.91 ± 0.03 | 30 | 30 | 694 |
| Out-of- | 3D | 0.010 | 0.717 | 0.010 | 0.037 | 0.580 | 0.200 | 0.941 | 10 | 8 | 529 |
| sample | IMRT | 1.401 | 0.805 | 0.025 | 0.014 | 0.544 | 0.273 | 0.727 | 10 | 10 | 1173 |
| (both types | SBRT | 1.926 | 0.465 | 0.010 | 0.075 | 0.503 | 0.141 | 0.875 | 10 | 7 | 724 |
| of SAs) | | | | | | | | | | | |

In Table 4, the f1 score was computed by averaging over 50 trials of random samples of the not-anomaly holdout set sₙ·f1 scores of 0.98 were found for 3D, 0.89 for IMRT, and 0.98 for SBRT, where the error bars run between 2-5%. For the feature switching generated simulated anomalies (SAs), f1 scores of 0.84 were found for 3D, 0.84 for IMRT, and 0.90 for SBRT with similar error bars, as shown in Table 4.

The model was also run on a training set, which was a combination of both Rx switched and feature switched SAs. The resulting f1 scores for the combined training set were found to lie in between the scores for the training sets where each type of anomaly was considered separately. The results and parameters are reported Table 4. Because the standard deviation is small, any run was chosen as the final parameters. Note that 0 and t varied slightly because of the different historical holdout samples.

Here, out-of-sample indicates that the distance model was run with the same parameters that were found by optimization over the training set, on a new unseen test set. E.g., in the test set, both the normal non-anomalous test records and the anomalous test records are previously unknown to the distance model. The distance model parameters for the out-of-sample runs were determined from the training/in-sample runs.

A separate, recent, data set (Jan. 1, 2021-Jul. 14, 2021) was used to select samples for the out-of-sample testing non-anomalous class data. All of the samples during this time period were used for the 3D and SBRT, each one containing ten samples. Ten of the most typical cases were selected out of the 24 IMRT samples from this time period as the testing normal class. For the out-sample case, the historical data set (from Jan. 1, 2006-Dec. 31, 2021) is still an important input into the model, however no samples are drawn from it for prediction.

A new set of anomalies was created for each technique using several construction methods, which served as the out-of-sample testing anomaly class data. Again, the anomalies were synthesized using several construction methods and the anomaly status was verified by looking at the conditional feature distribution after switching/changing features. The results are reported in Table 4. Comparing the out of sample performance to the in-sample, the out-of-sample is worse for IMRT and SBRT but better for 3D.

A beneficial feature of the distance model is that not only does it provide the model prediction for each of the test records, but it also provides an explanation why each prediction was made. By looking at the values of R, F, t_{F} and t_{Rx}, the reason why a sample was flagged or not flagged is immediately apparent.

### VI. Mock Peer Review

In order to compare the model performance with that of physicians in the real clinical setting, a mock peer review of the reduction to practice was conducted. Three radiation oncologists with more than ten years of experience treating thoracic patients at Johns Hopkins were each asked to independently label a sample dataset containing 17 anomalies and 30 normals (a subset randomly selected from out-of-sample testing data).

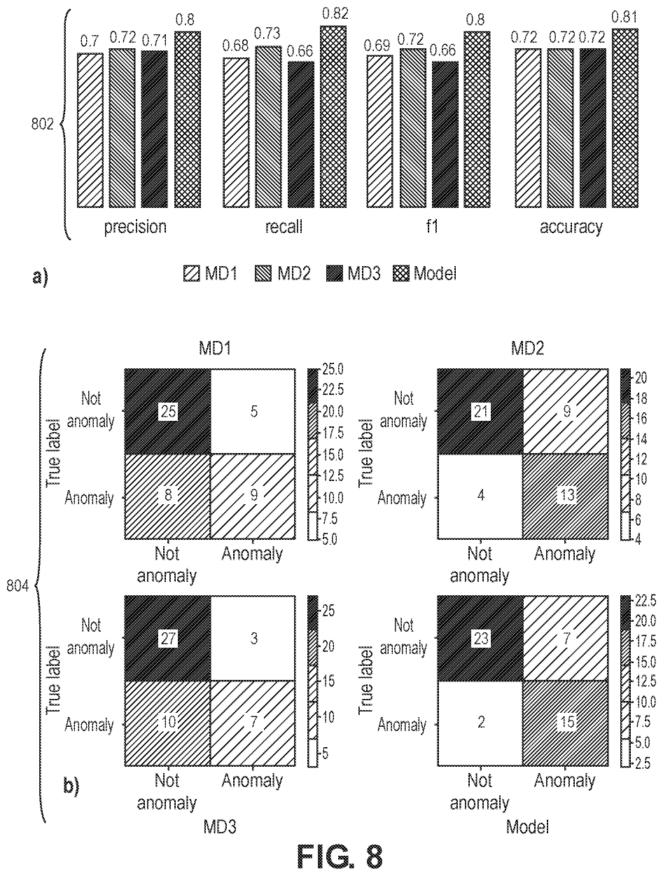

FIG. 8 depicts the results of the mock peer review. The results of the physicians, side-by-side with the model results are shown at 802. To get a sense of the time and effort spent by each physician on the mock peer review, each physician noted the time spent on the review. MD2 spent 18 minutes identifying the errors and 12 minutes to write out rationale. MD1 spent a total 11 minutes both identifying the errors and writing out the rationale for their decisions. The performance (a macro average of metrics) was evaluated by calculating f1, accuracy, precision and recall. The leftmost three bars of each of the four groupings indicate each MD's performance, and the rightmost bar in each of the four grouping indicates the model performance. The model's precision, recall, f1 and accuracy scores are all compatible with the MDs, suggesting that the model can serve a role as a digital peer.

Confusion matrices 804 for the MDs and the model are shown at 804. The confusion matrices 8045 give a breakdown of the different type I and type II errors made by each MD and the model. The model has the lowest false negative rate among the model and the MDs, suggesting that the model is more conservative than all the MDs in making the decision as to whether a case should be considered as an anomaly.

The model running time for a single testing sample is about 1 second, and the model training time is several days. However, the model may only be trained once, prior to deployment. The training time is proportional to the number of evaluation points in the grid space, the number of runs to average the ƒ1 score and the number of data samples.

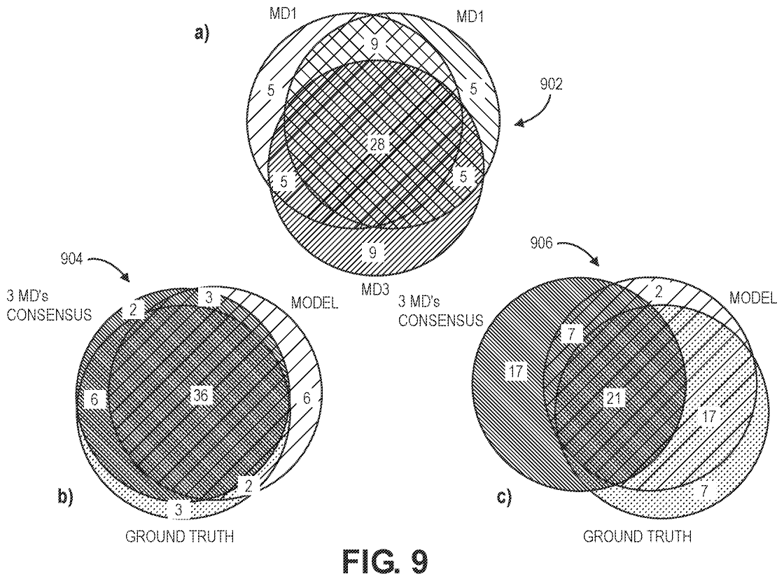

In the mock peer review, MDs were able to discuss each case and combine their knowledge in order to form a consensus about the correctness of a prescription for each case under review. Thus, in addition to comparing the performance of each MD individually against the model, them model is compared to the group consensus. A best and worst case scenario from joining were considered. In the worst case scenario, the peer review selects any incorrect decision from any of the three MDs as the consensus decision. If all three MDs predict correctly then the consensus decision is correct, otherwise the incorrect decision is chosen as the consensus decision. In the best case scenario, if any of the three MDs predict correctly, then that correct decision is taken as the consensus decision. If all three MDs predict incorrectly, then the consensus decision is taken as incorrect. It would be expected in the real clinical setting that the actual performance of peer review would lie somewhere between the worst case and best case consensus scenarios. The results of such a worst and best case joining of the MD decisions are displayed in FIG. 9 as well as the overlap diagrams of agreement for each individual MDs.

FIG. 9 shows Venn diagrams 902, 904, 906 illustrating the overlap of agreement between the individual MDs, the MDs' consensus, the model, and the ground truth. Diagram 902 shows the overlap of agreement between the three MDs on decisions of whether to flag or not to flag a particular case. Diagram 904 shows the best case scenario from the peer review, and diagram 906 shows the worst case scenario. Note that the numbers in the Venn diagrams 902, 904, 906 do not distinguish between anomalous or non-anomalous class. Any overlap regions with the ground truth set correspond to correct decisions, any decisions outside the ground truth set correspond to incorrect decisions.

In the worst-case scenario, represented by the diagram 906, the model outperformed the consensus by missing 9 (2+7) cases rather than 24 (17+7) cases by the consensus. The performance of the reduction to practice model was between those of the best and worst-scenarios, but closer to the former. The overlapping regions/agreements indicates that the model independently agreed with physician's knowledge.

The results of FIG. 9 should not necessarily be interpreted to suggest that the model underperformed or outperformed the MDs in the mock peer review. Rather, the results indicate that the model can be considered as an additional “digital peer reviewer” to complement the MDs. Under these circumstances, the distance model has promise as a validation tool to check for prescription errors since the model caught anomalies that the physicians did not notice.

### VII. Conclusion

One of the advantages of the reduction to practice model compared to a supervised learning model is that the distance model does not present any problem with class imbalance. This is due to the fact that the distance model is not a supervised learning model in the traditional sense, and instead relies on distances between historical data and the test set to define outcomes. When comparing the performance of the model versus the MDs' performance, even with the same level of performance, the model is still valuable because it is a fully automated process that does not require valuable physician time and provides an additional safety check.

Other approaches to anomaly detection typically use statistical methods such as joint probability density fitting or clustering methods. The approach of the reduction to practice is superior to, for example, the k-means clustering method because it does not perform any clustering of the data. Instead, it utilizes finding the “closest” neighboring data points in the feature space. Further, it is superior to k-nearest neighbors methods as well; for example, it does not rely on a simple voting scheme.

Thus, the reduction to practice has advantages over supervised learning models that are not a good fit for anomaly detection.

Certain embodiments can be performed using a computer program or set of programs. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), and magnetic or optical disks or tapes.

While the invention has been described with reference to the exemplary embodiments thereof, those skilled in the art will be able to make various modifications to the described embodiments without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

What is claimed is:

1. A method of detecting that a radiotherapy prescription for a patient is anomalous, the method comprising:
accessing a plurality of historical patient data for a plurality of historical patients, each historical patient datum comprising a historical radiotherapy prescription and a historical set of diagnostic features;
determining, by an electronic processor, at least one of a first measure or a second measure, wherein the first measure comprises a distance between the radiotherapy prescription represented as a point in a first multidimensional space and a historical radiotherapy prescription represented as a point in the first multidimensional space, and wherein the second measure comprises a distance between a set of diagnostic features of the patient represented as a point in a second multidimensional space and a historical set of diagnostic features, for a historical patient with a similar historical radiotherapy prescription, represented as a point in the second multidimensional space;
detecting, by the electronic processor, at least one of the first measure exceeding a first threshold or the second measure exceeding a second threshold; and
issuing an alert, by the electronic processor, in response to the detecting.

2. The method of claim 1, further comprising treating the patient according to a replacement prescription for the radiotherapy prescription.

3. The method of claim 1, wherein the first measure comprises an average of distances between the radiotherapy prescription represented as a point in a first multidimensional space and multiple of the plurality of the historical radiotherapy prescriptions represented as points in the first multidimensional space.

4. The method of claim 1, wherein the second measure comprises an average of distances between the set of diagnostic features of the patient represented as a point in a second multidimensional space and multiple historical sets of diagnostic features, each for a historical patient with a similar historical radiotherapy prescription, represented as points in the second multidimensional space.

5. The method of claim 1, wherein the first threshold comprises an average of distances between pairs of the plurality of historical radiotherapy prescriptions represented as points in the first multidimensional space.

6. The method of claim 1, wherein the second threshold comprises an average of distances between pairs of historical sets of diagnostic features of the plurality of historical patients represented as points in the second multidimensional space.

7. The method of claim 1, further comprising:
generating simulated anomalous patient data, the simulated anomalous patient data comprising at least one of: a simulated radiotherapy prescription mismatched with a set of diagnostic features, or a simulated radiotherapy prescription that occurs in less than a predetermined proportion of the plurality of historical patient data; and
optimizing model parameters using the simulated anomalous patient data.

8. The method of claim 1, further comprising:
determining that an element of the radiotherapy prescription occurs in less than some predetermined proportion of the historical radiotherapy prescriptions for a same technique as the radiotherapy prescription; and
issuing an alert, by the electronic processor, in response to the determining that an element of the radiotherapy prescription occurs in less than some predetermined proportion of the historical radiotherapy prescriptions for a same technique as the radiotherapy prescription.

9. The method of claim 1, wherein the set of diagnostic features comprises: treatment technique, treatment energy, treatment intent, diagnostic code, morphology code, and patient age.

10. The method of claim 1, wherein the issuing the alert comprises one of: issuing an alert indicating that the radiotherapy prescription is unlike historical radiotherapy prescriptions, or issuing an alert indicating that the radiotherapy prescription is mismatched to the diagnostic features of the patient.

11. A system for detecting that a radiotherapy prescription for a patient is anomalous, the system comprising an electronic processor and non-transitory electronic persistent storage storing instructions that, when executed by the electronic processor, perform actions comprising:
accessing a plurality of historical patient data for a plurality of historical patients in non-transitory electronic persistent storage, each historical patient datum comprising a historical radiotherapy prescription and a historical set of diagnostic features;
determining, by the electronic processor, at least one of a first measure or a second measure, wherein the first measure comprises a distance between the radiotherapy prescription represented as a point in a first multidimensional space and a historical radiotherapy prescription represented as a point in the first multidimensional space, and wherein the second measure comprises a distance between a set of diagnostic features of the patient represented as a point in a second multidimensional space and a historical set of diagnostic features, for a historical patient with a similar historical radiotherapy prescription, represented as a point in the second multidimensional space;
detecting, by the electronic processor, at least one of the first measure exceeding a first threshold or the second measure exceeding a second threshold; and
issuing an alert, by the electronic processor, in response to the detecting.

12. The system of claim 11, wherein the first measure comprises an average of distances between the radiotherapy prescription represented as a point in a first multidimensional space and multiple of the plurality of the historical radiotherapy prescriptions represented as points in the first multidimensional space.

13. The system of claim 11, wherein the second measure comprises an average of distances between the set of diagnostic features of the patient represented as a point in a second multidimensional space and multiple historical sets of diagnostic features, each for a historical patient with a similar historical radiotherapy prescription, represented as points in the second multidimensional space.

14. The system of claim 11, wherein the first threshold comprises an average of distances between pairs of the plurality of historical radiotherapy prescriptions represented as points in the first multidimensional space.

15. The system of claim 11, wherein the second threshold comprises an average of distances between pairs of historical sets of diagnostic features of the plurality of historical patients represented as points in the second multidimensional space.

16. The system of claim 11, wherein the actions further comprise:
generating simulated anomalous patient data, the simulated anomalous patient data comprising at least one of: a simulated radiotherapy prescription mismatched with a set of diagnostic features, or a simulated radiotherapy prescription that occurs in less than a predetermined proportion of the plurality of historical patient data; and
optimizing model parameters using the simulated anomalous patient data.

17. The system of claim 11, wherein the actions further comprise:
determining that an element of the radiotherapy prescription occurs in less than some predetermined proportion of the historical radiotherapy prescriptions for a same technique as the radiotherapy prescription; and
issuing an alert, by the electronic processor, in response to the determining that an element of the radiotherapy prescription occurs in less than some predetermined proportion of the historical radiotherapy prescriptions for a same technique as the radiotherapy prescription.

18. The system of claim 11, wherein the set of diagnostic features comprises: treatment technique, treatment energy, treatment intent, diagnostic code, morphology code, and patient age.

19. The system of claim 11, wherein the issuing the alert comprises one of: issuing an alert indicating that the radiotherapy prescription is unlike historical radiotherapy prescriptions, or issuing an alert indicating that the radiotherapy prescription is mismatched to the diagnostic features of the patient.

20. A non-transitory computer-readable medium comprising instructions that, when executed by an electronic processor, configure the electronic processor to detect that a radiotherapy prescription for a patient is anomalous by performing actions comprising:
accessing a plurality of historical patient data for a plurality of historical patients, each historical patient datum comprising a historical radiotherapy prescription and a historical set of diagnostic features;
determining, by an electronic processor, at least one of a first measure or a second measure, wherein the first measure comprises a distance between the radiotherapy prescription represented as a point in a first multidimensional space and a historical radiotherapy prescription represented as a point in the first multidimensional space, and wherein the second measure comprises a distance between a set of diagnostic features of the patient represented as a point in a second multidimensional space and a historical set of diagnostic features, for a historical patient with a similar historical radiotherapy prescription, represented as a point in the second multidimensional space;
detecting, by the electronic processor, at least one of the first measure exceeding a first threshold or the second measure exceeding a second threshold; and
issuing an alert, by the electronic processor, in response to the detecting.
